(19) United States
(12) Patent Application Publication
(10) Pub. No.: US 2008/0275494 A1
(43) Pub. Date: Nov. 6, 2008

(54) EXTENDED DURATION REMOVABLE MEDICAL FILTER WITH TISSUE BARRIER

(76) Inventor: James A. Fleming, Bethlehem, PA (US)

Correspondence Address: RONALD L. HOFER, 122 LINDBERGH LANE, MOORESVILLE, NC, 28117, US

(21) Appl. No.: 11/742,752

(22) Filed: May 1, 2007

(51) Int. Cl. A61M 29/00

(52) U.S. Cl. 606/200

(57) ABSTRACT

A medical filter intended for placement inside a tubular body passage of a patient has a filter frame with a cell growth inhibiting sheet attached to the struts of the frame to prevent or minimize growth of cells over the frame after it has been implanted in a vessel or body passage of a patient.

### BACKGROUND AND SUMMARY OF THE INVENTION

The present invention relates to medical filters which are intended to be placed inside a blood vessel or other body passage for the purpose of intercepting thrombus or particles.

Medical filters, including vena cava filters, are emplaced inside blood vessels or other body passages for the purpose of intercepting thrombus, clots or other particles while allowing free flow of blood in the vessels or other body fluid in the body passages. A medical filter should be emplaced with minimal trauma to the patient as by percutaneous delivery, and if it is desired to remove the filter, it is desirable that it can be removed with minimal trauma to the vessel or body passage. Of course, it is also desirable that the filter be adapted to properly adjust to the size of the vessel, that it be stable within the vessel, and that the filter remain effective during its time in place.

Sometimes it is desirable to remove a medical filter such as a vena cava filter after it has been emplaced. However, such removal may encounter difficulties. For example, after a conventional vena cava filter with struts has been placed in vivo into the Inferior Vena Cava, the cells of the inner lining, or endothelium, begin to multiply and grow over any portion of the filter that is touching the caval wall. In order for the filter to be retrieved without considerable trauma, it must be removed before this tissue growth becomes so complete that it surrounds the struts of the filter and prevents its withdrawal from the cava.

Generally speaking, effective vena cava and other medical filters are known. For example, medial filters are disclosed in U.S. Pat. Nos. 6,443,972 Sep. 3, 2002 to Bosma et al. for “Vascular Filter” and 6,989,021 Jan. 24, 2006, to Bosma et al. for “Retrievable Medial Filter.” But there remains room for improved designs of medical filters. In particular, it would be desirable to have a filter designed to be implanted for a longer period of time than a conventional filter and yet be removable with minimum trauma to the vessel. Accordingly, the present invention is in the nature of an improvement of the aforementioned filters and provides a medical filter which is suitable to be implanted in a vessel for an extended period of time yet can be removed with minimal trauma to the vessel. The medical filter of this invention is resistant to migration and has enhanced filtering characteristics. Furthermore, the filter is well suited for implantation in curved vessels.

Thus, in accordance with the present invention, a medical filter intended for placement inside a tubular body passage such as a vessel of a patient comprises, in a radially expanded state:
a filter frame having a plurality of longitudinally extending ribs or struts and at least one end piece with a retrieval element; and
a plurality of sheets of cell growth inhibiting material attached to radially outwardly facing surfaces of said ribs.

In a preferred embodiment of a medical filter, the sheets of material comprise a biocompatible material with a surface finish that prevents the direct attachment of tissue growth to the tissue. In another preferred embodiment, the sheets of material comprise an absorbable fabric tissue. The design of the filter is such that it can be readily retrieved from a body cavity or vessel even after an extended duration in place. The design will easily collapse into a cylindrical shape that can be loaded into a sheath for removal.

Further understanding of the present invention will be had from the following description taken in conjunction with the accompanying drawings and appended claims.

### BRIEF DESCRIPTION OF THE DRAWINGS

FIG. 1 is a side elevation of a preferred embodiment of a medical filter of the present invention shown in expanded form;

FIG. 2 is an end elevation of the preferred embodiment of FIG. 1;

FIG. 3 is a side elevation of an alternative preferred embodiment of the present invention shown in expanded form;

FIG. 4 is an end elevation of the alternative preferred embodiment of the medical filter of the present invention;

FIG. 5 is a perspective view, broken away, illustrating a preferred embodiment of a medical filter of the present invention in partially drawn down form; and

FIG. 6 is a perspective view, broken away, illustrating another alternative preferred embodiment of a medical filter of the present invention employing sheet material in tubular form.

### DETAILED DESCRIPTION OF THE PREFERRED EMBODIMENTS

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the preferred embodiment of the present invention are shown in use in a vein but may also be used in other vessels or body passages.

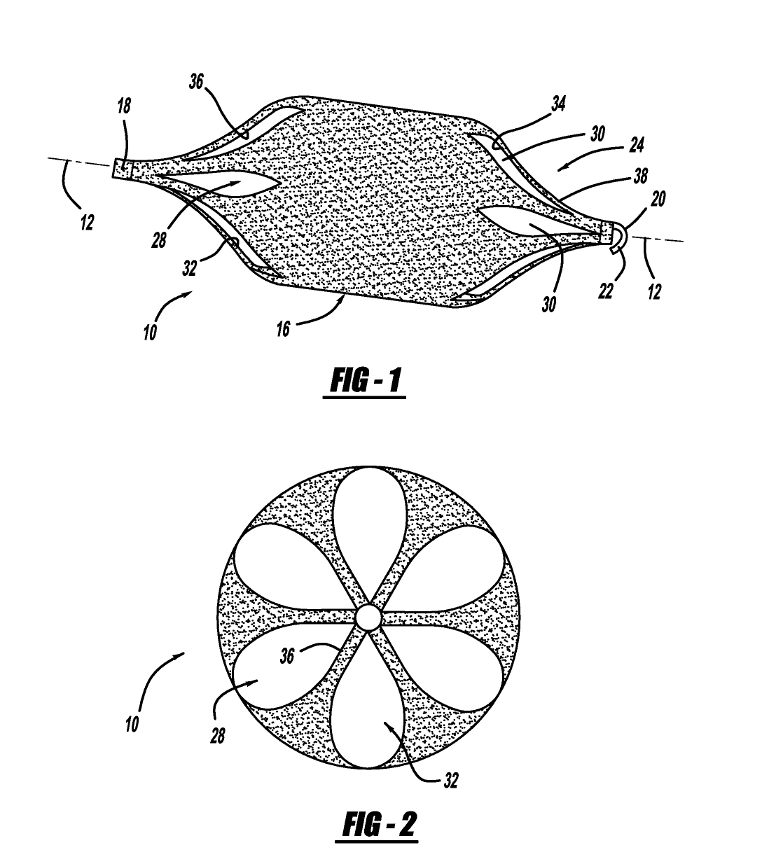

Now referring to FIGS. 1 and 2, a preferred embodiment of a medical filter of the present invention is shown and indicated generally by the numeral 10. Broadly speaking, medical filter 10 has an elongated shape with a longitudinal axis 12 and is compressible for insertion into and retrieval from a body passage but is shown in expanded form in FIGS. 1 and 2. Medical filter 10 broadly comprises filter frame 14 with sheet material 16 thereon as is described in more detail below.

Filter frame 14 has a pair of end pieces 18 and 20 located at opposite longitudinal ends of filter 10 and on its longitudinal axis. End piece 20 carries a conventional retrieval element 22. Extending generally radially outwardly and axially from each end piece 18 and 20 are a plurality of ribs 24 with parallel axially extending segments 26. Ribs 24 cooperate to form a pair of filter baskets with filter openings 28 and 30. Retrieval element 22 may be a single hook as shown in the figures or may have a T-shape with twin hooks or any suitable shape which can operatively interact with a snare device. It will be appreciated by those skilled in the art that hooks and snares are well-known and that the particular retrieval means employed is subject to variation within the spirit of the present invention.

Each arm rib 24 of filter frame 14 is made of a resilient material, preferably a shape memory material, which tends to expand to the form illustrated in FIGS. 1 and 2 but can be compressed radially to a smaller diameter to be carried in the lumen of a suitable delivery catheter. The filter frame 14 of filter 10 can be made of any suitable material using a variety of methods. Nitinol is a preferred material but elgiloy, cobalt chromium, stainless steel or suitable plastic are examples of other materials that may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter. Thus, the material must also be sufficiently resilient to accomplish both compression in the delivery catheter and expansion upon ejection from the catheter.

Suitable methods of manufacture of filter frame 14 of medical filter 10 include cutting a pattern into a tube to enable expansion of the tube into the desired body and struts. Another suitable method is forming the struts and body from separate strips or wires and then joining the respective parts together by suitable methods which are well known in the art.

Filter frame 14 may be subject to various modifications within the broad scope of the present invention. For example, the preferred embodiment shown in FIGS. 1 and 2 has ribs 24 equally radially spaced about end pieces 18 and 20, but it will be appreciated by those skilled in the art that the exact number of ribs 24 may vary within the scope of this invention and that that more or fewer ribs 24 and more or fewer filter openings 28 and 30 may be used. In general, suitable filter frames for use in the present invention are known in the art and include, for example, those shown and described in U.S. Pat. No. 6,443,972 B1 Sep. 3, 2002 to Bosma et al. for “Vascular Filter” and in U.S. Pat. No. 6,989,021 B2 Jan. 24, 2006 for Bosma et al. for “Retrievable Medical Filter”. The disclosures of both of the aforementioned patents to Bosma et al. are specifically incorporated by reference herein.

In one sense, the present invention is an improvement to already known filter frames such as those mentioned above. Thus, in accordance with the present invention, a sheet 16 of material is disposed on ribs 24 and segments 26 to be interposed between ribs 24 and the inner lining or endothelium of a vessel or body passage into which filter 10 is deployed. Sheet 16 can be attached or otherwise secured to ribs 24 by adhesives, stitching or by any other suitable means.

Sheet 16 may be a single sheet with apertures 32 and 34 therein to allow flow of fluid through filter openings 28 and 30. Alternatively, a plurality of sheets 16 may be used and positioned so that openings are provided for flow of fluid to be filtered. Sheets 16 are comprised of a thin material which does not allow ingrowth of the endothelium. Any biocompatible material with a surface finish, coating, or structure that prevents the direct attachment of tissue growth from the associated vessel or body passage wall is suitable for use herein. For example, an absorbable fabric tissue barrier material such as INTERCEED® product from Johnson and Johnson may be used. Alternatively, sheet 16 may be coated with Heparin or other pharmaceutical agent that prevent or delay tissue/endothelium growth and/or thrombus formation. Preferably, sheet 16 has tapered portions 36 and 38 to prevent endotheilium from reaching any portion of the filter rib and to maintain an open flow path.

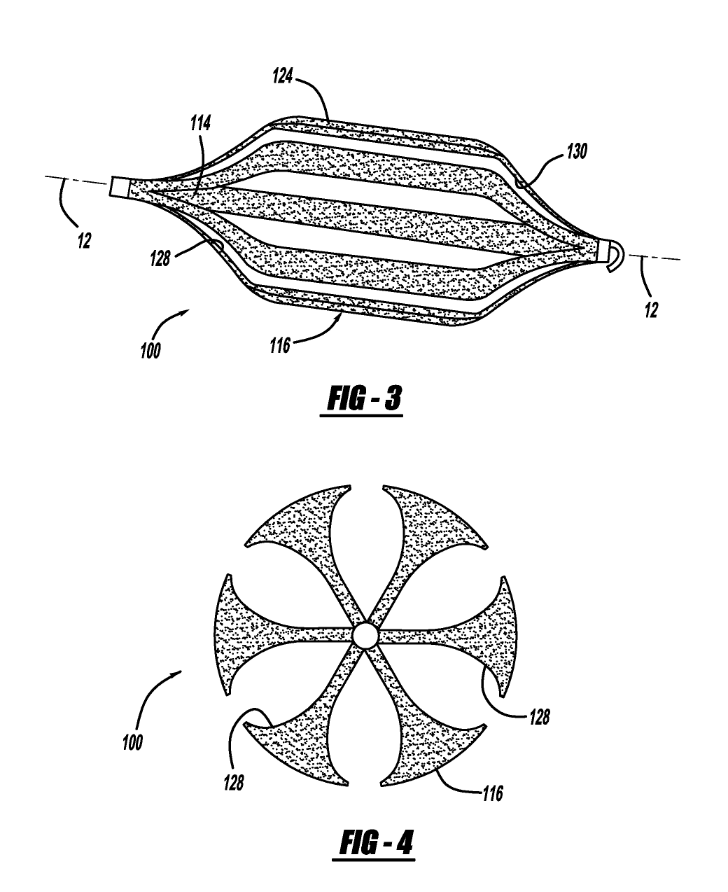

Of course, a variety of shapes of sheets 16 are suitable for use in the present invention. Now referring to FIGS. 3 and 4, an alternative embodiment of a medical filter of the present invention is illustrated and indicated by the numeral 100. Filter 100 has a filter frame 114 which is of a construction analogous to that of frame 14 of filter 10 and will not be described in further detail herein. However, filter 100 has sheets 116 of cell growth inhibiting material which are of a different shape than that of sheet 16 but serve the same effective purpose. Thus, filter 100 has a plurality of sheets 116 covering ribs 124 of filter frame 114 to prevent or minimize growth of cells over ribs 124. Filter 100 has filter openings 128 and 130 allowing suitable flow of fluid through filter 100 with filtration thereof.

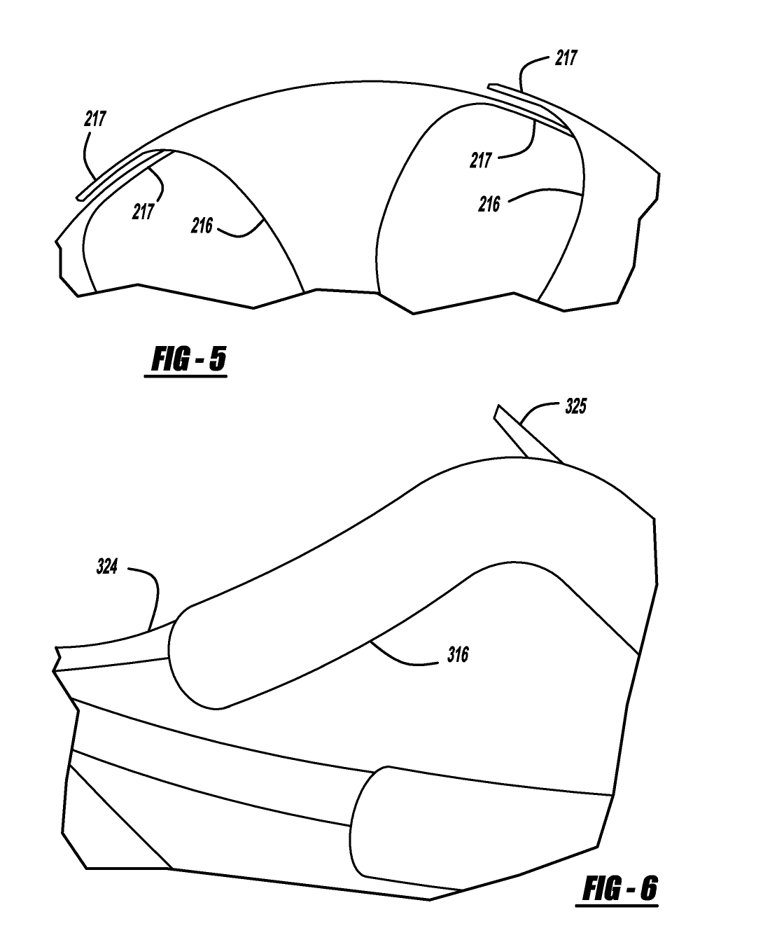

Still further variations of sheets 16 are shown in FIGS. 5 and 6. FIG. 5 illustrates a plurality of sheets 216 with overlapping edge portions 217. This arrangement facilitates radially collapsing and expanding filter 200 during its insertion and removal, which operations are generally conventionally carried out with an insertion catheter and removal catheter. FIG. 6 illustrates tubular sheets 316 place about ribs 324. FIG. 6 also illustrates hooking elements 325 carried on ribs 324 and extending radially outwardly beyond sheets 316.

Insertion or implantation of a filter of the present invention is done in a conventional manner as is well known in the art using an insertion catheter or the like.

While the medical filter of the present invention is suitable for permanent placement in a vessel or other body passage, the improvement of this invention relates to the facilitation of its removal or retrieval, which action is facilitated because cell growth about the filter frame is eliminated or minimized by use of sheets of cell growth inhibiting material. Retrieval of the medical filter may be accomplished by means of a conventional retrieval catheter which may be inserted from the appropriate direction toward the end of the filter having a retrieval element. The snaring or other engagement of the snare or the like of a retrieval catheter is accomplished in a conventional manner and then the filter is drawn into the lumen of the retrieval catheter.

While preferred embodiments of the present invention have been specifically described above, it will be appreciated by those skilled in the art that the present invention is subject to variations and modifications. For example, the filter may be cut from a single tube and have arms which are integral, i.e., one piece, with the spine. The filter may be tube-based or wire based or a combination. The curvature of the arms may vary. The number of arms and/or filter elements may vary. The size, configuration or number of sheets may vary. These and other modifications are contemplated to be within the scope of the present invention which is intended to be limited only by the following claims.

What is claimed is:

1. An elongated medical filter for placement inside a tubular body passage of a patient comprising:
(A) a filter frame extending along the longitudinal axis of said filter, said frame carrying a retrieval element at one end thereof,
(B) a cell growth inhibiting sheet attached to said frame and positioned radially outwardly on said frame.

2. The medical filter of claim 1 wherein said frame has at least one end piece at one end of said frame and on the longitudinal axis of said filter and said frame further comprises a plurality of ribs extending generally radially outwardly and longitudinally from said end piece.

3. The medical filter of claim 1 wherein said sheet is coated with a cell growth inhibiting agent.

4. The medical filter of claim 1 wherein said sheet is coated with Heparin.

5. The medical filter of claim 1 wherein each said sheet is comprised of an absorbable fabric tissue barrier material.

6. The medical filter of claim 1 wherein said filter has a plurality of openings in said sheet to allow flow of fluid therethrough.

7. The medical filter of claim 1 comprising a plurality of said sheets of cell growth inhibiting material.

8. The medical filter of claim 7 wherein said sheets are coated with a cell growth inhibiting agent.

9. The medical filter of claim 8 wherein said agent is Heparin.

10. The medical filter of claim 7 wherein said sheets are comprised of an absorbable fabric tissue barrier material.

11. The medical filter of claim 7 wherein said sheets have overlapping edge portions when said filter is in a radially compressed state.

12. The combination of a body vessel of a patient and implanted in said vessel, an elongated medical filter comprising:
(A) a filter frame extending along the longitudinal axis of said filter, said frame having at least one end piece at one end of said frame and on the longitudinal axis of said filter and said frame further comprising a plurality of ribs extending generally radially outwardly and longitudinally from said end piece, said frame carrying a retrieval element at one end thereof;
(B) a cell growth inhibiting sheet attached to said frame and positioned radially outwardly on said frame.

13. The combination of claim 12 wherein said sheet is coated with a cell growth inhibiting agent.

14. The combination of claim 12 wherein said sheet is coated with Heparin.

15. The combination of claim 12 wherein said sheet is comprised of an absorbable fabric tissue barrier material.

16. The combination of claim 12 wherein said filter has a plurality of said sheets of growth inhibiting material.

17. The combination of claim 16 wherein said sheets have overlapping edge portions when said filter is in a radially compressed state.

18. The combination of claim 16 wherein said sheets are coated with a cell growth inhibiting agent.

19. The medical filter of claim 18 wherein said agent is Heparin.

20. The medical filter of claim 16 wherein said sheets are comprised of an absorbable fabric tissue barrier material.
